(19) United States
(12) Patent Application Publication
(10) Pub. No.: US 2019/0192015 A1
(43) Pub. Date: Jun. 27, 2019

(54) Apparatus And Method For Auscultation

(71) Applicant: WITHINGS, Issy Les Moulineaux (FR)

(72) Inventor: David Campo, Issy Les Moulineaux (FR)

(73) Assignee: WITHINGS, Issy Les Moulineaux (FR)

(21) Appl. No.: 16/225,627

(22) Filed: Dec. 19, 2018

(30) Foreign Application Priority Data
Dec. 22, 2017 (EP) 17306914.7

(51) Int. Cl. A61B 5/021; A61B 5/00

(52) U.S. Cl. CPC A61B 5/02141; A61B 5/7203; A61B 5/6824; A61B 2562/04; A61B 5/0006; A61B 2562/0204; A61B 5/74

(57) ABSTRACT

An apparatus (1) comprising a cuff configured to be worn around an upper arm of a user (75), a first sensor (S1) comprised in the cuff and configured to receive sounds through a side of the chest of the user, and a second sensor (S2) configured to receive sounds and to be movable with regard to the cuff and configured to be placed at a front side of the user's chest and used in conjunction with the first sensor.

### FIELD OF THE DISCLOSURE

The present disclosure relates to apparatuses and methods for auscultation.

### BACKGROUND OF THE DISCLOSURE

Heart auscultation is of great interest since some of heart disorders can be detected non-invasively by analysing characteristic sounds emitted by the heart cyclic operation.

There are known auscultation devices with one stethoscope head and with a filtering function seeking to eliminate background noises from the heart sounds.

However, there remains a need to provide improved methods and systems to enhance analysis of heart sounds and detection of heart conditions.

### SUMMARY OF THE DISCLOSURE

There is disclosed an apparatus comprising a cuff configured to be worn around a upper arm of a user, a first sensor (S1) attached to an external wall of the cuff and having a sensitive side oriented away from the axis of the cuff and configured to receive sounds through a side of the chest of the user, and a second sensor (S2) configured to receive sounds and to be movable with regard to the cuff and configured to be placed at a front side of the user's chest and used in conjunction with the first sensor.

The clause ‘oriented away from the axis of the cuff’ is to be interpreted as ‘oriented away from the internal wall of the cuff’.

There is provided a two-sensor apparatus that can be used by a user himself/herself, the first sensor being placed against the side of the chest at a repeatable location. For most people this is the left side of the chest but a small minority are dextrocardiac which means the heart is on the right side of the chest. The second sensor is placed by hand of the user. Thereby, the apparatus receives signals representative of heart sounds sensed a two different locations at the user's chest and it will be possible to use these two signals to improve analysis of heart sounds.

It should be noted that the above apparatus can be used in a medical facility or at home, or in any environment, for self-auscultation either independently or with help of e.g. telemedicine services. The user can be a patient or an individual taking care of his/her health. The apparatus can be used for both diagnosis of illnesses and preventive healthcare. The first sensor is mounted in the cuff, and therefore it favours a natural placement of the sensor against the side of the chest as will be explained later.

The use of two distinct stethoscope heads for heart auscultation notably allows to distinguish very short sounds which can be indicative of some suspected medical condition in on or more heart valves.

We note that the promoted apparatus can be used by a patient alone, without the presence of any medical professional.

Further, one of the following features can be used alone or in combination.

According to an example embodiment, the first sensor (S1) may be an acoustic sensor. The first acoustic sensor may be a first stethoscope head. This first stethoscope head may be used for auscultation. This first sensor may be provided in the arm cuff and therefore, it is easy for the user to correctly place the first sensor by placing the cuff around his/her upper arm, and then placing the arm against his/her chest.

According to an example embodiment, the second sensor (S2) may be an acoustic sensor. The first acoustic sensor may be a second stethoscope head. This second stethoscope head may be used for auscultation. This second sensor is movable and can be handled easily and conveniently by one hand of the user.

According to an example embodiment, the first sensor (S1) may be fixedly mounted in the cuff. This first sensor may be housed in the arm cuff and therefore, it is easy and straightforward for the user to correctly place the first sensor by placing the cuff around his/her upper arm.

According to an example embodiment, the second sensor (S2) may be detachably mounted in the cuff. Whereby, when the apparatus is to be stowed, the second sensor can be attached to the cuff in a convenient manner When in use, the second sensor is detached to be moved around.

According to an example embodiment, the arm cuff may comprise at least a pneumatic bladder for blood pressure measurement.

The apparatus can house circuity for a blood pressure measurement function.

In such case, the apparatus can perform a blood pressure measurement function together with an auscultation function. Whereby, monitoring of bio parameters can be enhanced.

According to an example embodiment, the second sensor and the cuff may be connected with a wired connection. A very reliable connection is thereby provided, insensitive to possible electromagnetic pollution or jammers. Wire shielding can be provided to reject extraneous electric noises. Privacy of signals is also ensured.

According to an example embodiment, the second sensor and the cuff may be wirelessly connected. This is a very convenient and user-friendly solution, the second sensor can be moved around freely without taking care of any physical connection. This configuration also allows to place the second sensor under clothing very easily since there is no physical connection between the second sensor and the cuff.

According to an example embodiment, the apparatus can be connected to a display device such as a smartphone, a tablet or a computer. Thereby, instructions, feedback, results of analysis, and possibly other notices can be given to the user. Also, user profile and personal history can be handled through the display device.

According to an example embodiment, the display device is configured to provide guidance on positioning the second sensor. It is therefore possible to guide the user such that the second sensor is positioned right in the interval between two ribs. This leads to improved signal-to-noise ratio and improvement of the quality of the signal coming from the second sensor.

According to an example embodiment, the apparatus may further comprise a control circuitry which comprises a noise cancelling circuitry which is configured to reduce noise from signals received from at least one of the first or second sensors. The noise cancelling circuitry may use signals from the first and/or signals from the second sensors. The control unit is able to compare signals from the first and second sensor and to eliminate partially or totally extrinsic noises, for example common mode noises, to output a denoised signal. Thereby it is possible to improve the quality and accuracy of signals indicative of sounds emitted by the heart of a user or patient.

According to an example embodiment, the apparatus the may be configured to be connected to one or more remote server(s), directly or through the display device. Therefore, the apparatus can be used in connection with telemedicine services. Analysis of a possible medical condition can be therefore performed by trained medical personnel, and/or with the help of a medical expert system.

According to an example embodiment, the apparatus can be configured to receive an ECG signal (ECG stands for electrocardiogram). Timings of heart sounds can be je to electrical signals coming from the heart. According to an example embodiment, an ECG function can be integrated in the apparatus.

According to an example embodiment, there may be provided two or more electrodes arranged either at an internal wall of the cuff and/or at the first sensor and/or at the second sensor. Thereby, mechatronic integration is improved.

According to an example embodiment, the apparatus can comprise a housing arranged adjacent to the cuff, the housing enclosing a control unit. Protection and strength are provided by a such housing.

According to an example embodiment, the apparatus can comprise a vibrator, for giving a haptic feedback to the user. Thereby, the user can be notified accurately when to stop moving the second sensor S2.

According to one aspect of the present disclosure, it is disclosed a method comprising:

/a/—receiving acoustic signals from a first sensor (S1) arranged at a cuff configured to be worn around an upper arm of a user (75),

/b/—receiving acoustic signals from a second sensor (S2) configured to be movable with regard to the cuff and placed at a front side of the user's chest,

/c/—analysing acoustic signals sensed by the first sensor and by the second sensor. There may additionally be provided:

/d/—analysing the signals sensed by the first and second sensors for providing correction instructions to correct the placement of the second sensor (S2) thereby providing guidance for positioning the second sensor.

In particular, such correction instructions and guidance can help the user for positioning the second sensor at one of the prescribed heart auscultation positions (P1, P2, P3, P4, P5) at a front side of the user's chest.

Whereby, the first sensor is at a steady location, with a generally even signal level, whereas by contrast, while the second sensor is moved, the signal level received at the second sensor is affected by the presence of the ribs. Reference given by the first sensor provides guidance to help the user to place the second sensor properly at one of the prescribed positions.

According to one particular option, the method may further comprise:

sending first set of data to a remote server and receiving a second set of data from the remote server. The remote server may be part of a telemedicine service. Thereby, telemedicine can be involved to provide either correction instructions for placement of the second sensor and/or to provide diagnosis.

According to one particular option, the method may further comprise:

/e1/—signalling to the user that a pre-defined position for the second sensor is reached,

/e2/—processing the signals of the first sensor and/or the second sensor with a noise cancelling algorithm. Thereby, effective signal analysis starts after correct placement of first and second sensors and analysis is carried out with a stable configuration.

According to one particular option, the method may further comprise:

/d2/—providing correction instructions graphically on the display device coupled to the auscultation apparatus. Thereby, notice and guidance is given in an intuitive manner

According to one particular option, the method may further comprise:

/d3/—providing feedback to notify proper position (for example by way of visual feedback, and/or audio feedback, and/or haptic feedback). Thereby, the user knows accurately when to stop moving the second sensor S2.

According to one particular option, the method may further comprise:

/e/—carrying out analysis of signals at a telemedicine service and sending back health information such as a diagnosis. Thereby, the apparatus can be used at home by an individual without the presence of any medical personnel and can be supported by remote telemedicine service(s) to get back health information such as a diagnosis.

### BRIEF DESCRIPTION OF THE DRAWINGS

Other features and advantages of the disclosure appear from the following detailed description of one of its embodiments, given by way of non-limiting example, and with reference to the accompanying drawings, in which:

FIG. 1 illustrates a general overview of an apparatus according an example embodiment in a use configuration,

FIG. 2 shows a diagrammatic sectional view of a cuff of the apparatus in place on the left arm of the user, and adjacent to the chest, and second sensor at the front of the chest,

FIG. 3 shows an illustrative block diagram of the apparatus according to a first example embodiment,

FIG. 4 shows a timeplot illustrating an example of the noise cancelling feature,

FIG. 5 shows a diagrammatic view of a chest skeleton, heart and locations of auscultation

FIG. 6 shows a timeplot illustrating the method at a larger timescale, when placing the second sensor,

FIG. 7 illustrates a general flowchart of data processing,

FIG. 8 shows an illustrative block diagram of a variant of the apparatus according to a second embodiment, having a blood pressure sensing function, and optionally a ECG function,

FIG. 9 shows a general timeplot illustrating the method, at the heartbeat timescale,

FIG. 10 shows a timeplot illustrating a detection of aortic stenosis,

FIG. 11 illustrates a general overview of the disclosed method,

FIGS. 12A and 12B illustrate a detachable sensor.

### DETAILED DESCRIPTION OF THE DISCLOSURE

In the figures, the same references denote identical or similar elements. For clarity purposes, some parts are represented intentionally not at scale with regard to other parts. Also, some parts of timeplots can be represented intentionally not at scale.

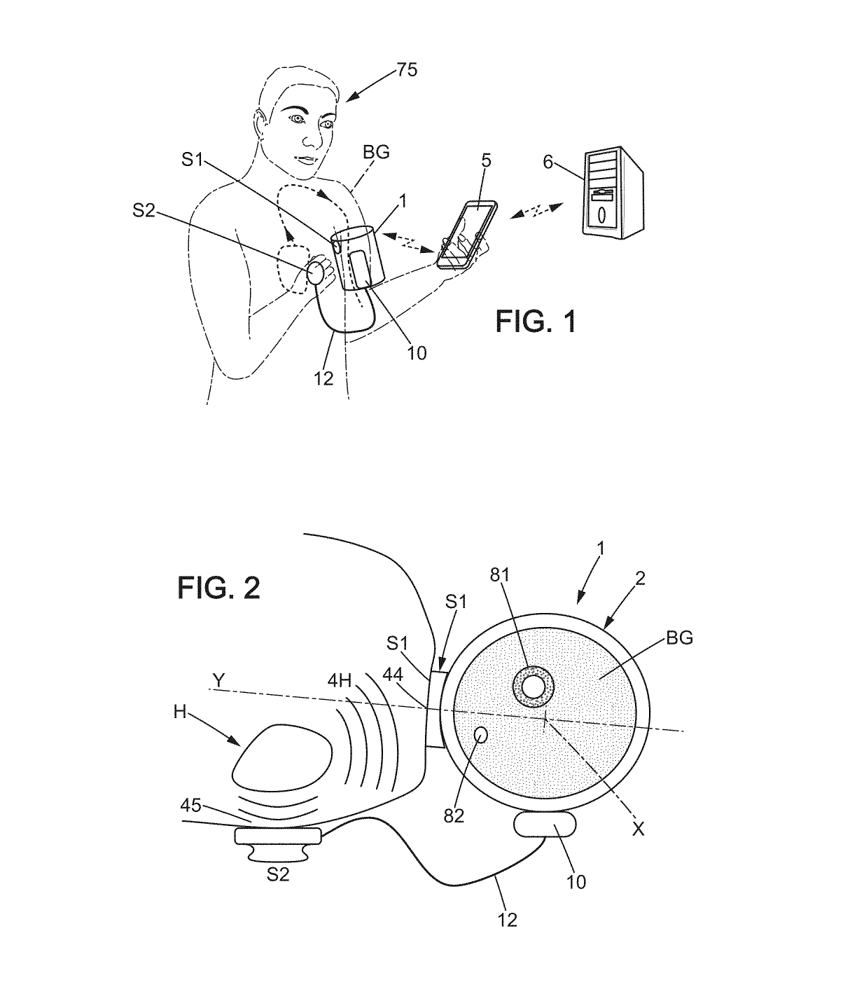

As shown in FIGS. 1 and 2, an apparatus 1 according to an example embodiment comprises a cuff 2, a first sensor S1 and a second sensor S2. The first sensor S1 can be an acoustic sensor. The second sensor S2 can be an acoustic sensor.

In an example embodiment, the first sensor S1 is a first stethoscope head.

In an example embodiment, the second sensor S2 is a second stethoscope head.

The apparatus 1 is an auscultation apparatus. In an example embodiment, the apparatus 1 is configured for heart auscultation.

The cuff 2 is formed as an arm band to be worn around an upper arm of a user 75. As illustrated, the cuff is wrapped around the upper left arm of the user 75. There is provided a housing 10 for housing a control unit that will be discussed later.

In one example of an embodiment, the cuff 2 is an arm band with clipping means and/or serrating means to be wrapped tightly and steadily around the upper arm.

In another example of embodiment, the cuff 2 may carry additional function(s) like blood pressure monitoring and optionally electrocardiogram (ECG) function.

The left arm BG of the user includes a bone named the humerus 81, muscles (not shown), and the brachial artery 82. The cuff 2, when wrapped around the arm BG, has a general cylindrical shape with a reference axis substantially coinciding with the arm axis.

The first sensor S1 is comprised by the cuff, whereas the second sensor S2 is configured to be movable with regard to the cuff. In other words, the distance between first and second sensors S1,S2 is not fixed, said distance is adjusted according to the patient morphology and the second sensor target location.

By the clause “comprised by the cuff”, it shall be understood the first sensor can be mounted in the cuff or placed adjacent to the cuff itself as non-limiting examples.

By the clause “mounted in the cuff”, it shall be understood the second sensor can be for example received and housed in a recess of the cuff in a detachable manner; the first sensor can be attached to the cuff, in a fixed manner Other ways to mechanically associate the first sensor or the second sensor to the cuff can be envisioned.

In the shown example, the first sensor S1 is attached to the external wall of the cuff. According to one example the first sensor S1 is fixedly mounted in the cuff in a predetermined position, without possibility to move the first sensor S1 with regard to the cuff. In such case, once the user has positioned the cuff correctly, the position of first sensor is correct.

According to another example, the first sensor S1 can be placed at two or three predetermined positions, according to the type/gender of user or according to the size of the user's arm.

The first sensor S1 has a sound receiving face 44 oriented away from the cuff axis (i.e. away from the arm). In the shown example, the first sensor is slightly projecting outwards; however alternately, the sensor can be fully integrated in the cuff without significant protrusion.

In use, the first sensor S1 is placed against the side of the chest (left side for most people) at a repeatable location, and the second sensor S2 is placed by hand of the user (here by the right hand). The height along the arm can be determined with reference to the biceps muscle, or with reference to the elbow joint.

In the shown example, the user holds a display device 5 in the left hand. In the shown example the display device 5 is a smartphone. The purpose of the display device will be discussed later.

The user 75 may be a patient or any individual wishing to monitor one or more of his/her bio-parameters. The apparatus can be used for both diagnosis of illnesses and preventive healthcare.

The apparatus can be used in medical facility or at home, or in any environment, for self-auscultation either independently or with help of medical personnel and/or telemedicine services.

In the shown example, the second sensor is coupled via a wire 12 to the cuff 2. More precisely the wire is connected at one of its end to the control unit housing 10 and at the other end to the second sensor such as second stethoscope head. The length of the wire may be comprised between 25 cm and 60 cm.

The second sensor S2 is detachably mounted in the cuff. When in use, the second sensor is detached from a stowed position to be moved around. When the apparatus is to be stowed, the second sensor can be attached to the cuff at the stowed position.

The stowed position can be defined as a position along the external wall of the cuff, where there may be provided a recess for receiving and maintaining there the second sensor S2.

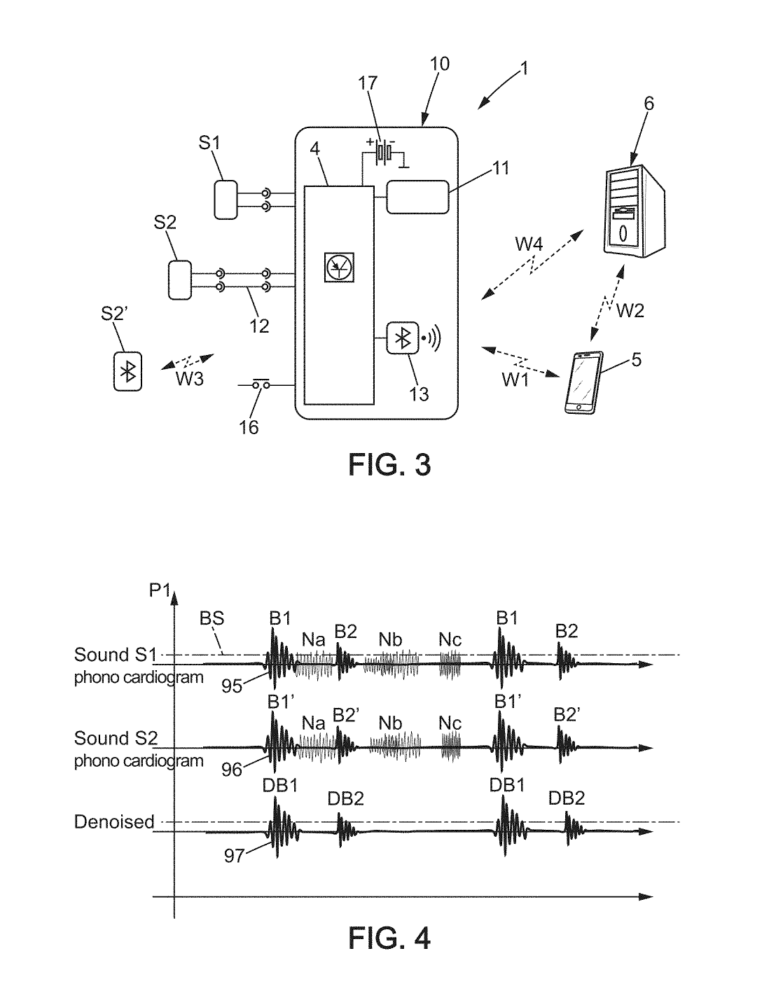

According to a variant, instead of the wire, a wireless link W3 is used between the second sensor S2′ (FIG. 3) and the control unit 4.

The apparatus 1 has a wireless communication capability W1 to exchange data with a display device 5 such as a smartphone or other mobile device like a tablet or a laptop.

Such display device 5 may in turn exchange data with a remote entity like a remote server 6 more generally any resource accessible via a network connection, for example via the Internet, through a data link W2 (wireless+internet). According to another configuration, there is provided a direct communication W4 between the apparatus 1 and the remote server 6.

Cyclic operation of the heart H generates sound waves 4H which propagate in all directions across tissues.

The first sensor S1 which is interposed between the left side of the chest and the arm equipped with the cuff has a sound receiving face 44 configured to receive the sound waves. The first sensor S1 can be positioned on bare skin, or can be positioned on a piece of clothing. Interposition of clothing does not significantly decrease the sensed signal and signal quality is sufficient even if the first sensor is applied on a piece of underwear, shirt, T shirt, or the like.

The second sensor S2 is movable with regard to the cuff, and is configured to be positioned at the front side of the chest. The positions of auscultation for the second sensor S2 will be discussed below. Under this condition, the second sensor S2 has a sound receiving face 45 placed against the chest and configured to receive sound waves from the heart H. The second sensor S2 can be positioned on bare skin, or can be positioned on a piece of clothing; indeed an interposition of a clothing does not significantly decrease much the sensed signal and signal quality is sufficient even if the second sensor S2 is applied on a piece of underwear, shirt, T shirt, or the like.

As shown in FIGS. 3 and 8, the housing 10 comprises a control unit 4.

The control unit 4 receives signals from the first sensor S1 and the second sensor S2. There may be provided a battery 17 serving as power source. There may be provided a local display 11. There is provided a wireless transceiver 13 (Bluetooth™, WiFi, or any similar solutions). There is provided a switch 16.

The control unit 4 comprises a control circuitry which may comprise a noise cancelling circuitry.

According to a first embodiment (FIG. 3, FIG. 4), the apparatus 1 can be used for auscultation. The apparatus 1 can be used for auscultation of an animal or a human individual.

The example use cases described above concern heart auscultation. However, other use cases are possible. Lung auscultation can also be carried out with the apparatus 1.

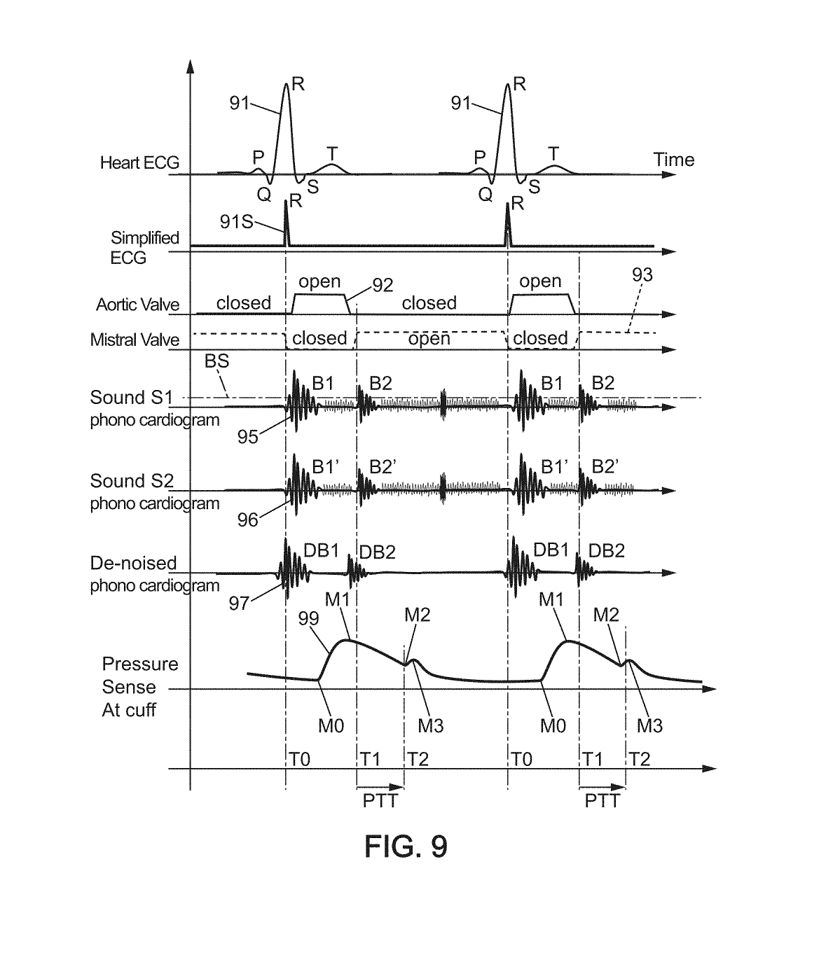

According to a second embodiment (FIG. 8, FIG. 9), the apparatus 1 has additional features. The apparatus 1 may be provided with a blood pressure sensing function and/or an ECG function. These two additional functions will be described later.

Returning to the first example embodiment as depicted at FIGS. 3,4,7, the control unit 4 is configured to analyze a first signal 95 received from the first sensor S1 and a second signal 96 received from the second sensor S2.

Phonocardiograms representing the first and second signals 95, 96 are shown at FIG. 4. A phonocardiogram (PCG) is a graphic representation of heart sounds.

Noise Cancellation Algorithm

The sounds picked up at the first sensor Si and the second sensor S2 can be affected by various noises Na, Nb, Nc at FIG. 4.

Such noises Na, Nb, Nc for heart auscultation can come from lungs, bowels, movement of the user, cloth rubbing, or even from remote devices like air conditioning, conversations nearby, telephone ringing, door banging, aircraft passing by, drilling works in the building, etc..

The control unit is configured to use an algorithm to suppress noises and keep intrinsic sounds of the heart.

The control unit 4 is configured to perform one or more correlation calculation from the first and second signals 95,96 coming from the first and second sensors S1, S2.

Subtracting signals picked up at the first sensor S1 from the second sensor S2 allows to eliminate or reduce common mode signals which affect both sensors similarly.

Further, the noise cancellation algorithm can be based on a Wavelet analysis.

According to a variant the noise cancellation algorithm can be based on a FFT analysis.

According to one option, digital filtering can be used, to eliminate or reduce high-frequency signal components.

A noise cancellation algorithm can be applied to first signal alone, or to second signal alone. A noise cancellation algorithm can be applied in a combined manner to first and second signals.

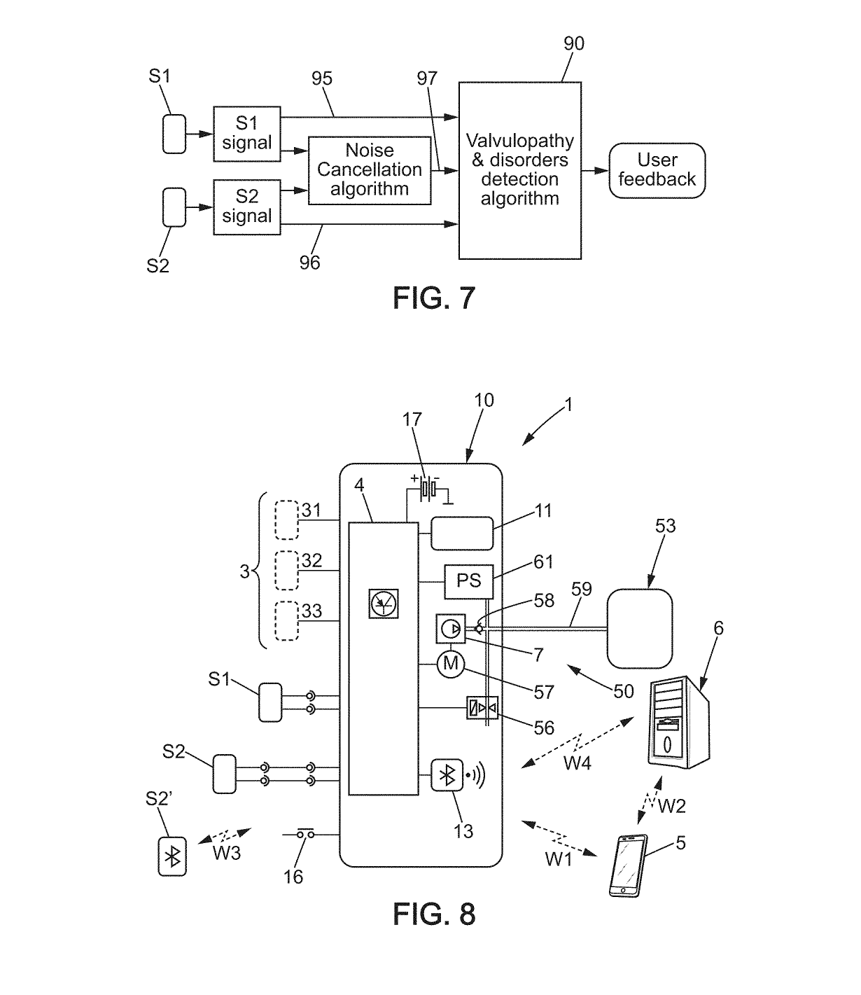

At the output of a noise cancellation block described at FIG. 7, a third signal 97 is available. In one example, the third signal 97 is a low noise signal, since most the noise has been removed from the first signal and/or the second signal by the noise cancellation block.

As shown at FIGS. 4 and 9, the phonocardiogram representing the first signal 95 exhibits two characteristics sounds; the first sound B1 corresponds to the closing of the mitral valve, the second sound B2 corresponds to the closing of the aortic valve. The phonocardiogram representing the second signal 96 exhibits similar characteristics sounds; B1′ corresponds to the closing of the mitral valve and tricuspid valve, B2′ corresponds to the closing of the aortic valve and pulmonary valve.

DB1 and DB2 correspond to denoised sounds, result of the above-mentioned algorithms

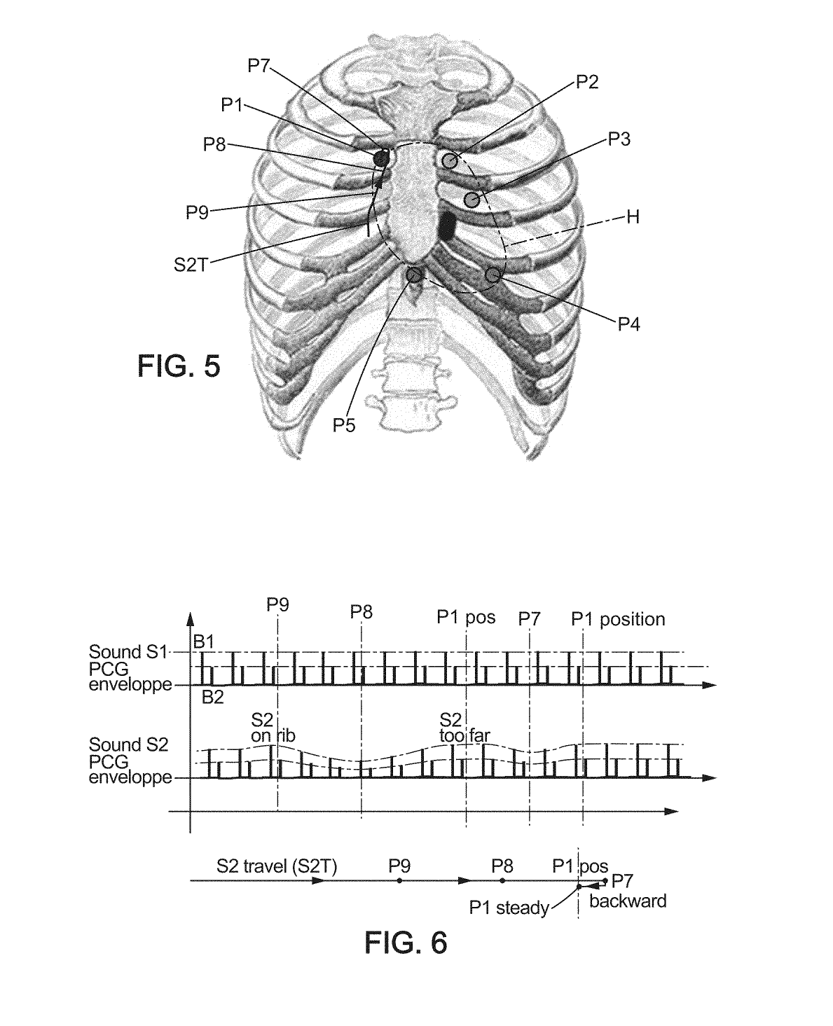

Referring now to FIG. 5, medical practice defines some prescribed heart auscultation positions P1, P2, P3, P4, P5, as illustrated at FIG. 5.

P1 is known as aortic area: right second inter costal space

P2 is known as pulmonic area: left second inter costal space

P3 is known as “Erb's point”: left third inter costal space

P4 is known as mitral area: left fifth inter costal space,

P5 is known as tricuspid area: left lower sternal border

In an example embodiment, the apparatus 1 provides correction instructions to correct the placement of the second sensor S2 at one of the prescribed heart auscultation positions (P1, P2, P3, P4, P5) at a front side of the user's chest, as illustrated at FIG. 6.

When the user moves the second sensor S2 along the travel S2T, the output of the first sensor S1 is not affected by movement of the second sensor S2 and therefore the envelope of the signal transduced by first sensor S1 is flat. By contrast, the envelope of the signal transduced by second sensor S2 fluctuates according to the presence or not of a rib or sternum interposed between the second sensor S2 and deep tissues (e.g. heart).

As illustrated, at position P9, the amplitude envelope is high, whereas by contrast at position P8, the amplitude envelope is low. The target position here is P1.

The analysis of the envelope of the signal transduced by second sensor S2 allows the control unit 4 to give a notice to the user either to go on moving or to stop moving or to go back.

P7 position is a bit too far since the amplitude envelope has decreased.

When it is determined that the position P1 has been reached, the user is notified for example with a message “stop moving” and “keep steady”.

FIG. 7 summarises the data flow, wherein functional blocks are shown, with functionalities partially or totally performed by the control circuitry of the control unit. There is provided an analysis block 90 which comprises a valvulopathy and disorders detection algorithm. Firstly, the first signal 95 is analysed individually; secondly the second signal 96 is analysed individually, and the third signal 97 issued by the noise cancellation algorithm is also analysed in conjunction with the two first ones.

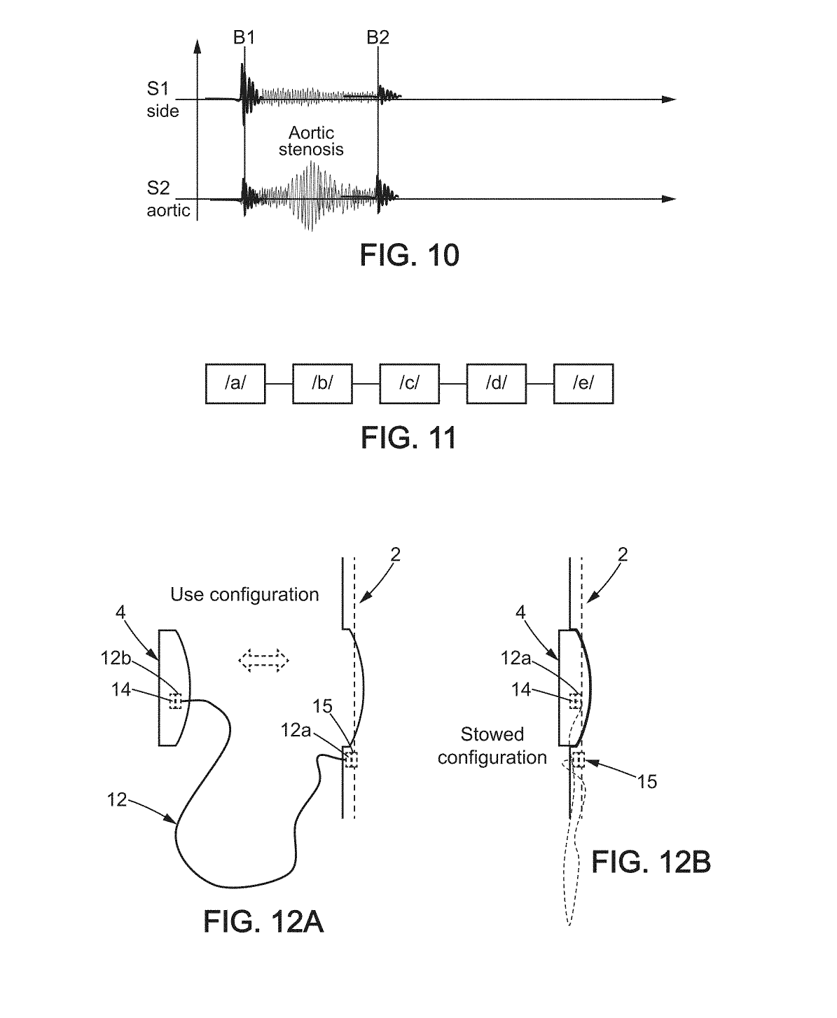

The algorithm in the analysis block 90 outputs a report with unexpected noises, such as noises being characteristic of a valve disorder. One example of a heart disorder, aortic stenosis, is given in FIG. 10, without any intended limitation.

In one embodiment, the algorithm in the analysis block 90 is fully implemented locally in the control unit 4.

In a variant embodiment, the algorithm in the analysis block 90 is implemented partly locally in the control unit 4 and partly in the display device 5.

In another variant embodiment, the algorithm in the analysis block 90 is implemented partly locally in the control unit 4 and partly in the remote server 6.

In another variant embodiment, the algorithm in the analysis block 90 is implemented partly locally in the control unit 4, partly in the display device 5 and partly in the remote server 6.

In another variant embodiment, the analysis block is implemented partly in the display device 5 and partly in the remote server 6.

The second sensor S2 can be of a wired type or of a wireless type (S2′).

As illustrated at FIG. 12A, in the case of the wired type, together with the cuff 2, there may be provided the above-mentioned cable 12. At one end of this cable, there is provided a connector 12a counterpart of the base connector 15; at the other end, there is provided a connector 12b counterpart of the second sensor connector 14. The cable 12 can comprise two wires. More wires are not excluded.

The cable 12 can be detached for stowage. In another embodiment, the cable can remain and be wrapped or stowed against the cuff (see FIG. 12B).

According to an extended functionality variant of the device, the device comprises a blood pressure sensing function at the cuff.

There is provided a pneumatic unit 50 with an inflatable bladder 53 partly or totally around along the cuff.

The pneumatic unit 50 comprises at least a pump 7 which may be driven by an electric motor 57, a release valve 56, and a pressure sensor 61.

According to a particular embodiment, the inflatable bladder 53 is connected to the pneumatic unit by an integrated pneumatic connector, or otherwise via a tube 59.

The pneumatic unit 50 may optionally comprise a check valve 58. The release valve 56 may be an on/off valve or a proportional valve.

The housing 10 comprises a switch 16. The switch 16 can be a press switch, a capacitor switch or a touch switch. The switch can have an on/off function, a ‘start cycle’ function, without excluding auxiliary functions using short press and long press feature.

There can be provided also a tap actuation function using an embedded accelerometer.

The user may start a blood pressure measurement, after having installed the armband, by actuating the switch 16.

The device is powered by a battery 17. The battery is for example a rechargeable battery. The battery can be a lithium ion battery.

For a blood pressure measurement session, the control unit 4 is configured to first inflate the inflatable bladder 53 until blood flow is greatly reduced by the pressure exerted on the arm. During inflation, the analysis of the evolution of pressure signals allows to infer the systolic pressure and the diastolic pressure. The controller is configured to then progressively deflate the inflatable bladder 53. The progressive reinstatement of the blood pressure waves is also analyzed by the control unit 4 to infer the systolic pressure and the diastolic pressure, in confirmation or replacement of values deduced during the inflation phase.

Also, by using one or two of the acoustic sensor(s), it is possible to calculate the pulse transit time (PTT), as explained later.

According to an extended functionality variant of the device, the device comprises a ECG function.

In this configuration, there are provided ECG electrodes 31,32,33 collectively denoted 3. In an embodiment, at least two of the ECG electrodes are arranged in the inwardly facing wall of the cuff. There may be another one arranged at the external surface of the housing 10. In variant configurations, one of the ECG electrodes is arranged at the first sensor S1, and one of the ECG electrodes is arranged at the second sensor S2.

As illustrated at FIG. 9, waves accompany each heartbeat, and the heartbeat generates electrical waves that propagate through the body at a first speed and the heartbeat generates a pressure wave in artery network that propagates through the vasculature at a second slower speed.

Immediately after the ventricular contraction, the pressure wave leaves the heart and aorta, passes through the subclavian artery, to the brachial artery along the path P.

The ECG electrodes 31,32,33 capture electrical signals which pass to an amplifier and a filtering circuit within the control unit 4. For example, a filtering circuit is provided before the signal is digitized and entered into the microcontroller.

Within the control unit 4, the ECG signals are processed with an analog-to-digital converter to form the digitized ECG waveform and then recorded together with the time of occurrence, namely instant TO. ECG waveform includes a characteristic part, i.e. “QRS waveform” or “QRS complex”.

A simplified ECG curve 91S is shown at the second curve of FIG. 9.

The acoustic waves are also band-pass filtered and amplified, for example after digitization. For example, a band-pass filter with cutting frequencies [0.5 Hz-1 kHz] is applied, either in the analog font end before digitization or applied to the digitized acoustic signal.

A QRS waveform is shown in the ECG signal 91 on timeplot ‘Heart ECG’ at FIG. 9. Instant T0 corresponds in the illustrative embodiment to R apex, but another timing characteristic can be taken alternatively.

Aortic valve open/close state is also shown, signal 92.

Mitral valve open/close state is also shown, signal 93.

Just before aortic valve opening, the mitral valve closes. This produces a particular sound which is reflected in the first significant sound B1 as shown on signal 95.

The second sound B2 corresponds to the closing of the aortic valve at time T1.

It should be noted that the three phonocardiograms representing the first signal 95, the second signal 96, and the third signal 97 are similar to what has been explained before regarding FIG. 4, and thus not repeated here.

Further, after closing mitral valve and opening aortic valve, ventricular volume decreases as blood is ejected to the aorta. At the same time ventricular pressure exhibits a rounded apex.

The pressure curve 99 exhibits three characteristics apexes. The first apex M1 is a maximum apex; the second apex M2 is a minimum local apex; the third apex M3 is a maximum local apex.

Besides M0 is the minimum value, just before the rise which is a consequence of the arrival of the pressure pulse at the arm.

The second apex M2 is a marker corresponding to arrival of the effect of the closure of aortic valve at the brachial artery within the arm band. M2 occurs at time T2. Time interval T2-T1 is referred to as pulse transit time PTT.

It should be noted that when using the ECG signal (either complete ECG signal 91 or simplified signal 91S), the control unit 4 benefits from an improved time reference, instead of relying only on the first sound B1.

It should be noted that measuring systolic pressure and diastolic pressure, and optionally pulse transit time PTT, provides complementary information besides the phonocardiograms that can be utilized by an expert system or medical staff, to provide a more complete and more reliable diagnosis.

Pressure curve analysis can reinforce detection of aortic valve disorder. An oscillometric signal normally has an expected characteristic shape, and an aortic valve disorder can be suspected or determined whenever the characteristic of an oscillometric signal does not meet that characteristic shape.

As illustrated at FIG. 11, the disclosed method may comprise the steps /a/, /b/, /c/, /d/ as mentioned earlier in the summary of the disclosure. Further, the disclosed method may comprise the step /e/ as mentioned earlier in the summary of the disclosure.

According to one embodiment step /d/ may comprise the steps /d2/ and/or /d3/ as mentioned earlier in the summary of the disclosure. According to one embodiment step /e/ may comprise the steps /e1/ and /e2/ as mentioned earlier in the summary of the disclosure.

Regarding the physical dimensions of the promoted device, the height of the apparatus 1 along its longitudinal axis X is less than 24 cm, preferably less than 20 cm, and more preferably less than 16 cm. The weight of the apparatus 1 is less than 700 grams, preferably less than 500 grams and more preferably less 300 grams.

The time needed to provide a feedback to the user from the start of measurement is less than 60 seconds preferably less than 30 seconds and more preferably less than 20 seconds.

1. An apparatus comprising:
a cuff configured to be worn around an upper arm of a user;
a first acoustic sensor attached to an external wall of the cuff and having a sensitive side oriented away from the internal wall of the cuff and configured to receive sounds through a side of the chest of the user; and
a second acoustic sensor configured to receive sounds and to be movable with regard to the cuff and configured to be placed at a front side of the user's chest and used in conjunction with the first sensor.

2. The apparatus according to claim 1, in which the first sensor is fixedly mounted in the cuff.

3. The apparatus according to claim 1, in which the second sensor is detachably mounted in the cuff.

4. The apparatus according to claim 1, in which the cuff comprises a pneumatic bladder for blood pressure measurement.

5. The apparatus according to claim 1, in which the second sensor and the cuff are connected with a wired connection.

6. The apparatus according to claim 1, in which the second sensor and the cuff are wirelessly connected.

7. The apparatus according to claim 1, in which the apparatus is configured to be connected to a display device.

8. The apparatus according to claim 7, the display device being configured to provide guidance on positioning the second sensor.

9. The apparatus according to claim 1, further comprising a control circuitry which comprises a noise cancelling circuitry which is configured to reduce noise from signals received from at least one of the first or second sensors.

10. The apparatus according to claim 1, configured to be connected to one or more remote server(s).

11. The apparatus according to claim 1, wherein the apparatus is configured to receive an ECG signal.

12. The apparatus according to claim 1, provided with two or more electrodes arranged at an internal wall of the cuff and/or at the first sensor S1 and/or at the second sensor S2.

13. The apparatus according to claim 1, further comprising a housing arranged adjacent to the cuff, the housing enclosing a control unit.

14. The apparatus according to claim 1, further comprising a vibrator, for giving a haptic feedback to the user.

15. A method comprising:
receiving acoustic signals from a first sensor attached at a cuff placed around an upper arm of a user and having a sensitive side oriented away from the cuff axis, receiving signals from a second acoustic sensor configured to be movable with regard to the cuff and placed at a front side of the user's chest;
using acoustic signals sensed by the first sensor in conjunction with and simultaneously with signals sensed by the second sensor.

10. The method according to claim 15, further comprising:
analysing the signals sensed by the first and second sensors for providing correction instructions to correct the placement of the second sensor thereby providing guidance for positioning the second sensor.

17. The method according to claim 15, further comprising:
signalling to the user that a pre-defined position for the second sensor is reached; and
processing the signals of the first sensor S1 and the second sensor S2 with a noise cancelling algorithm.

18. The method according to claim 15, further comprising: providing correction instructions graphically on a display device coupled to the auscultation apparatus.

19. The method according to claim 15, further comprising: providing feedback to notify proper position, by way of visual feedback, and/or audio feedback, and/or haptic feedback.

20. The method according to claim 15, further comprising:
carrying out analysis of signals at a telemedicine service and sending back health information including a diagnosis.
